(19) United States
(12) Patent Application Publication
(10) Pub. No.: US 2004/0030128 A1
(43) Pub. Date: Feb. 12, 2004

(54) Process for the preparation of a mixture of epsilon caprolactam and/or epsilon caprolactam precursors

(76) Inventors: Rudolf Philippus Maria Guit, Maastricht (NL); Mathieu Johannes Guillaume Notten, Stein (NL); Robert Pestman, Geldrop (NL)

Correspondence Address: PILLSBURY WINTHROP, LLP, P.O. BOX 10500, MCLEAN, VA, 22102, US

(21) Appl. No.: 10/381,279

(22) PCT Filed: Oct. 3, 2001
(86) PCT No.: PCT/NL01/00728

(30) Foreign Application Priority Data
Oct. 3, 2000 (EP) 00203435.3

(51) Int. Cl. C07D 201/02

(52) U.S. Cl. 540/538

(57) ABSTRACT

Process for the preparation of a mixture of ε-caprolactam and ε-caprolactam precursors by reductively aminating 5-formylvaleric acid and/or 5-formylvalerate ester(s) in water with hydrogen and an excess of ammonia in the presence of a hydrogenation catalyst, wherein the process is conducted in a reactor of which the inside reactor wall material is a material containing at most 8 wt. % nickel.

The invention relates to a process for the preparation of a mixture of ε-caprolactam and ε-caprolactam precursors by reductively aminating 5-formylvaleric acid and/or 5-formylvalerate ester(s) in water with hydrogen and an excess of ammonia in the presence of a hydrogenation catalyst.

ε-caprolactam precursors are here defined as ε-aminocaproate ester, 6-aminocaproic acid and 6-aminocaproamide and/or oligomers of these compounds. With reductive amination is meant reduction of an aldehyde into an amine in the presence of ammonia.

Such a process is known from WO-A-9835938. This publication describes a process to continuously prepare an aqueous mixture of ε-caprolactam and ε-caprolactam precursors by continuously contacting methyl-5-formylvalerate with hydrogen and an excess of ammonia in the presence of a ruthenium on titanium oxide carrier catalyst. In the examples, the process is performed in a Hastelloy C reactor vessel.

A disadvantage of this process is that the catalyst system gradually deactivates after some days of operation.

The object of the present invention is to provide a process in which catalyst deactivation is minimized or at least reduced.

This object is achieved in that the process is conducted in a reactor of which the inside reactor wall material is a material containing at most 8 wt. % nickel.

With the inside reactor wall is meant the reactor wall of which the surface is in contact with the reaction mixture.

Preferably, the material contains at most 6 wt. % nickel.

It has surprisingly been found that with the process of the present invention the catalyst deactivation after some days of operation does not occur or occurs to a lesser degree.

Another advantage is that in the process of the present invention, no or almost no corrosion of the inside reactor wall material takes place.

Without wishing to be bound to any particular theory, we believe that conventional used reactor equipment material corrodes into the reductive amination reaction mixture due to the fact that the reaction medium causes complexation of nickel (present in relatively high amounts of conventional used reactor equipment). As a consequence of this nickel complexation (hereafter referred to as nickel corrosion), other metals of the reactor wall material migrates into the reaction mixture and are deposited on the catalyst. We further believe that the decrease of the catalyst activity is mainly caused by deposition of corrosion metals, especially molybdenum from the reactor wall on the catalyst. Preferably, the inside reactor wall material contains less than 5 wt. % molybdenum and more preferably less than 4 wt. %. It was not expected that the reactor wall material would easily corrode into the reductive amination reaction mixture comprising an aminocaproic acid and an excess of ammonia, especially not when the process is performed in a reactor vessel constructed of corrosion resistant materials (like for example Hastelloy C® and stainless steel SS 316). This is the more so as no significant corrosion of the reactor wall (constructed from conventional used corrosion resistant material (like for example Hastelloy C)) takes place when the reactor is exposed to aqueous mixtures of 6-aminocaproic acid in the absence of ammonia and at the temperature and pressure of the reductive amination reaction, or when the reactor is exposed to aqueous mixtures of ammonia in the absence of 6-aminocaproic acid and at the temperature and pressure of the reductive amination reaction. Moreover, it was not to be expected that the use of a reactor wall material containing relatively high amounts of nickel (like for example a Hastelloy C reactor (containing more than 50 wt. % of nickel) or an austenitic stainless steel (such as for example SS 316 containing between 8 and 15 wt. % nickel)) would cause catalyst deactivation after some days of operation, because reductive amination reactions are usually carried out using a Raney nickel catalyst and it is known from WO-A-0014062 that the activity of a ruthenium on carrier reductive amination catalyst is increased when nickel is present as a further catalyst component. Reductive amination of 5-formylvaleric acid in the presence of a Raney nickel catalyst is for example described in U.S. Pat. No. 4,950,429.

The material should be able to sustain the reaction temperatures and reaction pressures. Examples of suitable materials to be used as inside reactor wall material in the process of the present invention are metals, selected from titanium, zirconium, niobium and tantalum; polymers like for example polytetrafluoroethime polymer (PTFE) or polyvinylidenefluoropolymer (PVDF); and metal alloys such as ferritic stainless steel material and duplex stainless steel material. Duplex stainless steels are steels characterized by a ferritic-austenitic structure, where the two phases have different compositions. Duplex stainless steel is for example described in U.S. Pat. No. 5,582,656, the disclosure of which is incorporated herein as reference. An example of a suitable duplex stainless steel material is the commercially available duplex stainless steel SAF 2205®. Another example is Duplex 1.4362 (X2CrNiN 22-4) containing less than 0.6 wt. % molybdenum. From a technical point of view, based on its corrosion resistance, a ferritic stainless steel material is preferred and the above mentioned pure metals are even more preferred. From an economic point of view, based on its cost price and the processability, the use of the above mentioned pure metals is preferred and the use of duplex stainless steel is even more preferred. The use of duplex stainless steel is the most preferred from a practical point of view, based on the combination of corrosion resistance, processability and cost price.

In one embodiment of the invention, the reductive amination reaction is performed in a reactor vessel of which the entire wall is constructed from a material containing at most 8 wt. % nickel. In this embodiment of the invention the use of a ferritic stainless steel material is preferred and the use of duplex stainless steel is even more preferred.

In another embodiment of the invention, the reductive amination reaction is performed in a reactor vessel of which the surface of the wall in contact with the reaction mixture (hereafter called the inside reactor wall) is covered with a material containing at most 8 wt. % nickel. Hereafter the covering of the surface of the reactor wall which is in contact with the reaction mixture is called lining. An advantage of lining the reactor is that the material of the lining in contact with the reaction medium can be independently chosen from the base material of the reactor. Suitable base materials for the reactor are then the conventially used austenitic corrosion-resistant stainless steel such as for example SS304 and SS316. In this embodiment of the invention, the use of a ferritic stainless steel is preferred and the use of a pure metal is even more preferred. Although no particular limitation is imposed on the thickness of the lining, a thickness of 0.5 to 30 mm is sufficient. Providing the lining material on the inside reactor wall is conducted according to known methods. As the manner of lining, it is preferable to form a film of the lining material on the surface of the base material. The film may be formed by any suitable method, for example by overlay welding cladding, loose lining or explosive bonding. Alternatively, the inside reactor wall is chromated. Chromation is conducted according to known methods of chromating metal surfaces for example using electrolytic deposition of chrome from chrome salt solution.

The 5-formylvalerate ester starting compound can be represented by the following general formula:

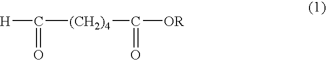

where R is an organic group with 1 to 20 carbon atoms, wherein the organic group is an alkyl, cycloalkyl, aryl or aralkyl group. More preferably R is an alkyl group. Examples of R groups include methyl, ethyl, propyl, isopropyl, n-butyl, tert-butyl, isobutyl, cyclohexyl, benzyl and phenyl. By preference R is methyl or ethyl. Preferably the starting compound is an alkyl 5-formylvalerate because these compounds are more readily available than 5-formylvaleric acid. Unless otherwise stated, reference herein to the formyl-starting compound means alkyl 5-formylvalerate, 5-formylvaleric acid, or both.

The reductive amination is performed by contacting the formyl-starting compound with the catalyst, hydrogen and an excess of ammonia in water. If the starting compound is a 5-formylvalerate ester it is preferred that some alcohol is present. The alcohol corresponding to the R-group of the 5-formylvalerate ester is preferred. More preferably, a water/corresponding alkanol mixture is used as solvent because the rate at which 5-formylvalerate ester is solved in these mixtures is increased compared to pure water. Water will be formed in the reductive amination step as a reaction product of the reaction between the formyl group of the alkyl formylvalerate compound and ammonia. The water content in the reaction mixture is at least 10 wt. %, more preferably between 15 and 60 wt. % and most preferably between 20 and 50 wt. %. The concentration of the alkanol is preferably between 1 and 25 wt. %.

The reaction mixture obtained in the reductive amination step comprises ε-caprolactam and ε-caprolactam precursors, ammonia, hydrogen, water and the corresponding alkanol.

The hydrogenation catalyst comprises at least one of the metals of Groups 8-10 of the Periodic System of the Elements (Handbook of Chemistry and Physics, 70th edition, CRC Press, 1989-1990). Preference is given to Ru-, Ni- or Co-containing catalysts. In addition to Ru, Co and/or Ni the catalysts can also contain other metals for example Cu, Fe, Rh, Pt and/or Cr. The catalytically active metals may be applied onto a carrier or not. Suitable carriers are for example aluminium oxide, silica, titanium oxide, zirconium oxide, magnesium oxide and carbon. Titanium oxide is preferably used as the carrier because of its high chemical and mechanical stability and because the selectivity to the preferred (intermediate) compounds is found to be relatively high when this support is used. Preferably anatase is used as titanium oxide. Non-supported metals can be used for example in the form of a finely dispersed suspension for example finely dispersed ruthenium. Preferred Ni- and Co-containing catalysts are Raney nickel and Raney Cobalt optionally in combination with small amounts of another metal, for example Cu, Fe and/or Cr. Most preferred are ruthenium containing catalysts.

Preferably, the hydrogenation catalyst is a ruthenium on titanium oxide carrier catalyst as for example described in WO-A-9835938. Optionally, the catalyst contains at least one further group 8-11 metal or compounds thereof as for example described in WO-A-0014062. Of the further group 8-11 metal Co, Rh, Ir, Ni, Pd, Pt and Cu are preferred. The most preferred further group 8-11 metal is Rh and Ni.

A relatively small but catalytically effective amount of the catalyst is used in the present process. The amount of the catalytically active metal (as metal) is generally between 0.1 and 10 wt %. If a further group 8-11 metal is present in the catalyst, its amount (as metal) in the catalyst (metals plus carrier) is generally between 0.05 and 30 wt. %, preferably between 0.1 and 10 wt. % and more preferably between 0.1 and 5 wt. %. The molar ratio of the catalytically active metal to the further metal is generally within the range from 100:1 to 1:10, preferably from 20:1 to 1:1. In case a supported catalyst is used, the mean particle size (d₅₀) of the catalyst is preferably between 10 and 100 μm, when the catalyst is present as a slurry in the reaction mixture or between 0.001 and 0.05 m, when the catalyst is present in a fixed bed. The BET surface area can be between 1 and 100 m²/g. The BET surface area is preferably between 30 and 100 m²/g. In case the carrier is chosen to be titanium oxide, preferably titanium oxide is used in its anatase form to reach such a high BET surface area of titanium oxide. The high BET surface area is advantageous because higher catalyst activity can be obtained.

The molar ratio of ammonia and formyl-starting compound in the reductive amination step is preferably between about 3:1 and about 30:1, and more preferably between about 5:1 and about 20:1.

The temperature is preferably between about 40° C. and about 200° C., and more preferably between about 80° C. and about 160° C.

The process is preferably conducted under pressure. In general, the pressure is equal or greater than the resulting equilibrium pressure of the liquid reaction mixture employed. The pressure is preferably between 0.5 and 12 MPa.

The molar amount of hydrogen is at least equal to the molar quantity of formyl-starting compound. The molar ratio of hydrogen to the formyl-starting compound is preferably between about 1 to about 100.

The reductive amination can be performed batch wise or continuously. A large scale commercial process will preferably be performed continuously.

In case a heterogeneous catalyst is used, the reductive amination can be performed continuously in a fixed bed reactor in which the heterogeneous hydrogenation catalyst is present. An advantage of this reactor is that the reactants are easily separated from the hydrogenation catalyst. Another manner of performing the reductive amination is by way of one or more continuously operated well mixed contactors in series in which the heterogeneous hydrogenation catalyst is present as a slurry (slurry reactor). This manner of operation has the advantage that the concentration gradients and the heat of the reaction can be easily controlled. Examples of specific and suitable slurry reactors are one or multiple staged bubble columns or a gas lift-loop reactor or a continuously stirred tank reactor (CSTR). The slurry-hydrogenation catalyst can be separated from the reaction mixture by for example using hydrocyclones, centrifuges and/or by filtration, for example by cake- or cross-flow filtration.

The catalyst concentration can be suitably selected across a wide concentration range. In a fixed bed reactor the amount of catalyst per reactor volume will be high, while in a slurry-reactor this concentration will, in general be lower. In a continuously operated slurry reactor the weight fraction of catalyst (including the carrier) is typically between about 0.1 and about 30 weight % relative to the total reactor content.

The 5-formylvalerate ester can be obtained by hydroformylation of the corresponding pentenoate as for example described in WO-A-9426688 and WO-A-9518089.

Subsequent to the reductive amination, the caprolactam precursors present in the reaction mixture can be further reacted to caprolactam as for example described in WO-A-9837063.

### EXAMPLE I

A continuous reductive amination experiment was conducted in a Hastelloy C microreactor which had been chromated (the baffles and impeller were provided with a lining of chromium by electrolytic deposition of chrome) and having a liquid volume of 25 ml. 1 gram 1.75 wt % ruthenium on titanium oxide (BET surface area 48 m²/g) was introduced in the reactor. An aqueous stream consisting of 40 wt % NH₃, 25 wt % methyl-5-formylvalerate and 7 wt % methanol in water was continuously fed to the reactor. The reaction was performed at a temperature of 140° C. and a pressure of 4 MPa. The liquid residence time was 1 hour. By operating the CSTR-type reactor at incomplete conversion, a change in the degree of conversion is a direct measure for the changing catalyst activity. The overall methyl-5-formylvalerate conversion to hydrogenated products was monitored by performing detailed chemical analysis of the reaction product mixture as a function of on-stream time. According to standard CSTR reactor theory, the apparent first order reaction rate constant (k) was calculated according to k=Degree of conversion/[Residence time*(1-Degree of Conversion)]. The reaction rate constant is a measure of the catalyst activity.

In Table 1 the reaction rate constant is given as a function of reaction time.

Comparative Experiment A

Example I was repeated with a Hastelloy C microreactor on which no chromation treatment was executed. In Table 1 the reaction rate constant is given as a function of reaction time.

TABLE 1
Reaction rate constant k (1/h) versus reaction
time (hours)

| | | k |
|---|---|---|
| | k | Comparative |
| Time | Example 1 | Experiment A |
| 0 | 4 | 4 |
| 109 | | 2.45 |
| 161 | | 2.12 |
| 252 | | 1.15 |
| 346 | | 0.74 |
| 399 | 2.08 | |
| 451 | | 0.56 |
| 499 | 1.45 | |
| 644 | 1.15 | |
| 763 | 0.86 | |
| 1002 | 0.56 | |
| 1268 | 0.45 | |

From Table 1 it can be seen that the deactivation in Comparative Experiment A is much faster than in Example I: In Example 1 k is reduced from 4 to 0.56 after 1002 hours, while in Comparative Experiment A k is reduced from 4 to 0.56 in only 451 hours.

### EXAMPLE II

A reductive amination reaction was carried out in a 1 liter baffled Hastelloy C reactor equipped with a turbine stirrer. Corrosion coupons of Hastelloy C-276 and of Duplex 1.4462 (Duplex X2CrNiMoN 22-5-3) were mounted on the baffles of this reactor in a galvanically insulated way. 60 grams of 5 wt % ruthenium on titanium oxide were introduced in the reactor. After the addition of water, the catalyst was pre-reduced at 140° C. during 12 hours. Subsequently, an aqueous stream of approximately 775 grams per hour, consisting of approximately 25 wt % methyl-5-formylvalerate, 35 wt % ammonia and 7 wt % methanol in water, was fed continuously to the reactor. The reactor was kept at a constant pressure of 4.0 MPa by a hydrogen stream of 10 grams per hour. The reaction was performed at 120° C. An average yield of desired products, i.e. ε-caprolactam and caprolactam precursors, of 97% was obtained. The corrosion coupons were exposed to the liquid reactor content of this experiment during 1082 hours.

After the experiment both corrosion coupons showed a smooth metal surface. From the weight-loss during the experiment (see Table 2 below) it was calculated that Hastelloy C-276 has a corrosion rate of 0.05 mm/year, while Duplex 1.4462 corroded at a rate of only 0.001 mm/year, showing that Duplex is a considerably more corrosion resistant material against the process conditions of the reductive amination process.

TABLE 2
Results of corrosion test

| | Area of the corrosion | Initial weight of | Weight of the | |
|---|---|---|---|---|
| | coupon exposed to the | the corrosion | corrosion coupon | Corrosion |
| Corrosion | reactor content | coupon | after exposure | rate¹ |
| coupon | [cm²] | [gram] | [gram] | [mm/year]² |
| Hastelloy | 10.1 | 7.7523 | 7.7002 | 0.05 |
| C-276 | | | | |
| Duplex | 8.3 | 6.2355 | 6.2342 | 0.001 |
| 1.4462 | | | | |

¹Density of both materials is approx. 8 gram/cm³
²For the calculation a yearly exposure time of 8000 hours has been used

1. Process for the preparation of a mixture of ε-caprolactam and ε-caprolactam precursors by reductively aminating 5-formylvaleric acid and/or 5-formylvalerate ester(s) in water with hydrogen and an excess of ammonia in the presence of a hydrogenation catalyst, wherein the process is conducted in a reactor of which the inside reactor wall material is a material containing at most 8 wt. % nickel.

2. Process according to claim 1, wherein the inside reactor wall material contains at most 6 wt. % nickel.

3. Process according to claim 1 or 2, wherein the inside reactor wall material contains less than 5 wt. % molybdenum.

4. Process according to any one of claims 1-3, wherein the inert material is selected from titanium, zirconium, niobium, tantalum, ferritic stainless steel material or duplex stainless steel material.

5. Process according to any one of claims 1-4, wherein the entire reactor wall is constructed from a duplex stainless steel.

6. Process according to any one of claims 1-4, wherein the inside reactor wall is provided with a liner of titanium, zirconium, tantalum or niobium.

7. Process according to any one of claims 1-6, wherein the hydrogenation catalyst contains at least one Group 8-10 element of the Periodic system of the Elements as catalytically active metal.

8. Process according to claim 7, wherein the catalytically active metal is chosen from ruthenium, nickel or cobalt.

9. Process according to claim 8, wherein the catalytically active metal is ruthenium.

10. Process according to claim 9, wherein the hydrogenation catalyst is a ruthenium on titanium oxide carrier catalyst.
